(19) United States
(12) Patent Application Publication
(10) Pub. No.: US 2021/0398668 A1
(43) Pub. Date: Dec. 23, 2021

(54) SYSTEMS AND METHODS FOR PROVIDING TELEHEALTH SESSIONS

(71) Applicant: CLOVER HEALTH INVESTMENTS, CORP., Franklin, TN (US)

(72) Inventors: Calvin Chock, Danville, CA (US); Broderick Koo, Tuen Mun (HK); Jacky Chow, Hong Kong (HK); David Zhu, Hong Kong (HK); Kevin Holub, Newton, MA (US); Anthony Via, New York, NY (US); Molly Inglish, Atlanta, GA (US); Alina Hoffman, Brooklyn, NY (US); Tod Loebel, New York, NY (US); Maruyi Lu, Pinole, CA (US); Andrew Toy, Woodside, CA (US)

(21) Appl. No.: 17/352,299

(22) Filed: Jun. 20, 2021

Related U.S. Application Data
(60) Provisional application No. 63/041,689, filed on Jun. 19, 2020.

(51) Int. Cl. G16H 40/67; G16H 80/00; G16H 40/20; G16H 10/60; H04L 29/08; G06F 3/0482

(52) U.S. Cl. CPC G16H 40/67; G16H 80/00; G06F 3/0482; G16H 10/60; H04L 67/141; G16H 40/20

(57) ABSTRACT

Methods and systems establishing a telehealth session between a patient and a healthcare provider (HP) are disclosed herein. One of the systems includes: a HP-side application residing a HP-side device; a patient-side application residing on a patient's device; and a telehealth application residing on a server. During a telehealth session, the HP-side application is configured to: generate one or more graphical user interfaces (GUIs) that display patient-related data on the display of the HP-side device; enable the healthcare provider to edit, using the one or more GUIs, one or more portions of the patient-related data; and send the one or more edited portions of the patient-related data to the telehealth application on the server for storage.

### CROSS-REFERENCE TO RELATED APPLICATIONS

This application claims priority under 35 U.S.C. § 119(e) to U.S. Provisional Application No. 63/041,689, entitled “SYSTEMS AND METHODS FOR PROVIDING EASY ACCESS TELEHEALTH,” filed Jun. 19, 2020, the disclosure of which is incorporated herein by reference in its entirety for all purposes.

### FIELD OF INVENTION

The subject matter described herein relates to online medical systems, and without limitation, some embodiments relate to systems and methods for providing telehealth sessions.

### BACKGROUND

Telehealth, which is the distribution of health-related services and information over a communications network, has been gaining popularity with the increasing speed of networks, the efficacy of available teleconferencing tools, and, most recently, the need for social distancing. However, current solutions can be complicated and difficult to use. For instance, most online telehealth systems require a user to traverse multiple screens and databases just to access desired information. In addition to that, existing teleconference systems require additional set up, e.g., installation and testing of teleconferencing software and entry of an identification to a particular teleconferencing session (often requiring many digits). For many patients, particularly sick patients who are in need of immediate attention, existing telehealth systems do not satisfy their needs. Accordingly, an improved telehealth system would be desirable.

### SUMMARY

Described herein are systems and methods that provide live telehealth sessions. In one embodiment, a patient can use a general computing device interface, and with a click of a button or touch of screen, initiate communication (e.g., a real-time video conference) with a medical service provider (such as a physician) to obtain the needed health service.

In another embodiment, the medical service provider can initiate the communication to a patient. Further, the medical service provider is provided an interface (e.g., a graphical user interface) with access to one or more databases that contain historical information about the patient to augment the provider's ability to provide the desired telehealth service.

In one of the systems for establishing a telehealth session between a patient and a healthcare provider (HP), the system includes: a HP-side application executable on one or more processors of a first device; a telehealth application executable on one or more processors of a server; and a patient-side application executable on one or more processors of the second device.

The HP-side application is configured to display a first graphical trigger on a display of the HP-side device that when selected causes the HP-side device to send a connection request to communicatively connect with the patient over the telehealth session.

The telehealth application is configured to receive, from the HP-side device, the connection request to communicatively connect with the patient. And, in response to receiving the connection request from the HP-side device, the telehealth application is configured to determine a contact information of the patient-side device; and send instructions to the patient-side device based on the contact information of the patient-side device.

The patient-side application is configured to display a second graphical trigger on a display of the patient-side device in response to receiving the instructions from the telehealth application. The second graphical trigger, when selected, causes the patient-side application of the patient-side device to communicatively connect with the HP-side application of the HP-side device to establish the telehealth session. The HP-side application is further configured to: generate one or more graphical user interfaces (GUIs) that display patient-related data on the display of the first device during the telehealth session; enable the healthcare provider to edit, using the one or more GUIs, one or more portions of the patient-related data during the telehealth session; and send the one or more edited portions to the telehealth application.

In some embodiments, the HP-side application is further configured to: display a list of patients on the display of the first device, wherein each patient on the list of patients is selectable; receive a selection of the patient from the list of patients; send information relating to the selected patient to the telehealth application; and receive patient-related data of the selected patient from the telehealth application. The HP-side application can also be configured to: obtain identifying information of the HP; and receive a list of patients based on the identifying information of the HP.

In the above example system, the telehealth application is configured to establish the telehealth session between the HP-side and the patient-side devices in response to receiving information indicating that the second graphical trigger is selected. The patient-side application is configured to connect to a virtual waiting room hosted by the telehealth application on the server prior to the telehealth session with the HP-side application of the HP-side device is established by the telehealth application.

The telehealth application can queue the patient-side application until the patient's scheduled appointment time and to establish the telehealth session between the HP-side and the patient-side devices once scheduled appointment time is reached, and to request the healthcare provider, using the HP-side client, to confirm moving the patient from the virtual waiting room to the live telehealth session.

The patient-side application can also authenticate a health monitoring device; receive one or more types of health data from the health monitoring device during the telehealth session; and send the one or more types of health data to the second device. The patient-side application can display the one or more types of health data on the display of the HP-side device.

The HP-side application can also receive and display the one or more types of health data on the display of the HP-side device. In some embodiments, the patient-side application is configured to enable the HP-side application to remotely control the health monitoring device by receiving and forwarding commands to the health monitoring device.

Other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE DRAWINGS

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a chart illustrating a telehealth system in accordance with some embodiments of the present disclosure.

FIGS. 2-3 are example graphical user interfaces of the telehealth system in accordance with some embodiments of the present disclosure.

FIGS. 4A-4B are example telehealth invitations.

FIGS. 5-6 are example graphical user interfaces of the telehealth system in accordance with some embodiments of the present disclosure.

FIG. 7 is an example graphical user interface of a patient-side application of the telehealth system in accordance with some embodiments of the present disclosure.

FIG. 8 is an example graphical user interface of the telehealth system in accordance with some embodiments of the present disclosure.

FIG. 9 is a flow chart illustrating a process for establishing a telehealth session in accordance with some embodiments of the present disclosure.

FIG. 10 is a flow chart illustrating a process for controlling and sharing data of a third party device in accordance with some embodiments of the present disclosure.

FIG. 11 is an example system diagram of the telehealth system in accordance with some embodiments of the present disclosure.

### DETAILED DESCRIPTION

Described herein are example systems and methods for providing easy access telehealth. One challenge for both patients and physicians is the anxiety that comes along with installing, navigating, and configuring technology solutions to enable telehealth, e.g., a telemedicine session, which may include a direct video conference between a patient and health care provider, such as a nurse or physician.

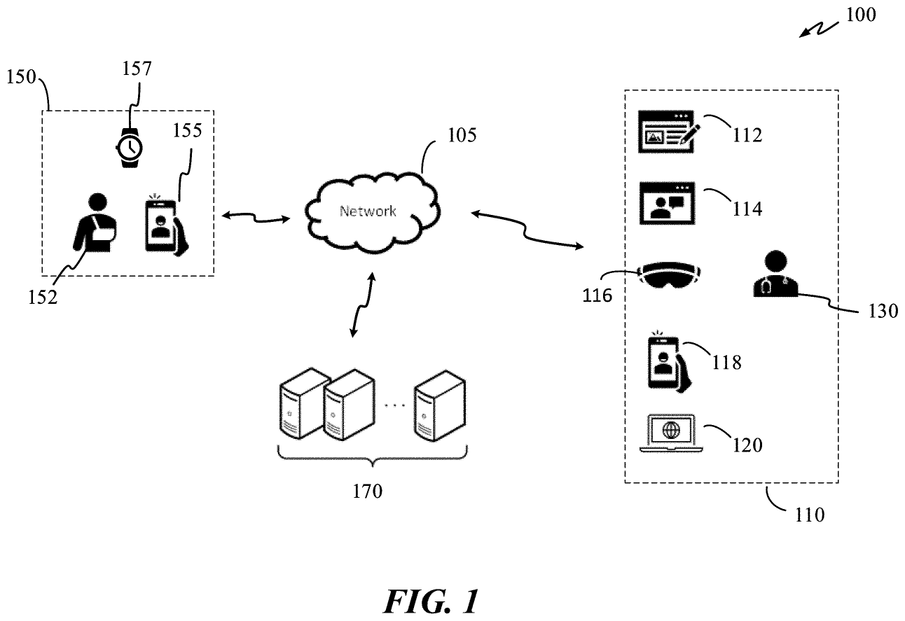

FIG. 1 illustrates an environment in which the system and methods for providing telehealth (hereinafter “telehealth system 100”) is implemented in accordance with some embodiments of the present disclosure. Telehealth system 100 includes a healthcare provider (HP) side 110, a patient side 150, servers 170.

HP-side 110 includes one or more telehealth applications that can connect to network 105. The one or more telehealth applications can reside on a computing device such as, but not limited to, a tablet 112, a display 114, an augmented/virtual reality device 116, a mobile device 118, and/or a laptop 120. Physician 130 can access patient data and/or medical resources from servers 170, via network 105. Servers 170 can be local or remote servers. Servers 170 can also include third-party data and/or services.

Patient-side 150 can include a patient-side telehealth application residing on the patient's computing device 155 (e.g., a mobile device, a desktop computer, a tablet). Computing device 155 can include one or more pre-existing client applications, such as a web browser (e.g., Microsoft Windows Internet Explorer, Mozilla Firefox, Apple Safari, Google Chrome, Opera), a short messaging service (SMS) application, an email application, or the patient-side telehealth application, which can be a widget, an extension, or an independent application. Patient-side 150 can also include one or more health-related devices 157 such as, but not limited to, heart rate monitors (e.g., mobile watch and phone with heart rate application), blood pressure monitoring devices (e.g., blood pressure cuff, phone, watch), Electrocardiogram (EKG) devices, Electroencephalogram (EEG) devices, body mass index measuring devices, and weigh scales. Device 157 can be communicatively coupled to device 155, which may require device 157 to be properly authenticated and qualified to provide certain services such as monitoring blood pressure, heart rate, breathing pattern, weight, temperature, etc.

In some embodiments, device 157 can be communicatively paired with device 155 using Bluetooth communication standard. Other communication standard can also be used such as, but not limited to, near field communication.

Server 170 can include one or more databases for storing patient-related data that can include personal information and health data including, but not limited to, medical history, diagnosis, prescription, and diet. Patient data may include personal information (e.g., age, gender, ethnicity, race, occupation, marital status, addresses), patient characteristics (e.g., hair color, eye color, shoe size, prosthetics, orthotics). Health data can also include medical history (e.g., previous diagnoses, medical procedures, surgeries, family medical history), laboratory results (e.g., blood panel), medical test results (e.g., echo stress test, EKG), pharmacological information (e.g., prescriptions, prescription fill information, preferred pharmacy), and healthcare information (e.g., insurance provider, member number, preferred doctor, home clinic). Server 170 can also include medical provider data such as, but not limited to, provider location information (e.g., office locations, address, accepted insurances, medical group), names and credentials of medical providers associated with a medical office and/or location, clinical visit times (e.g., average time, preferred time, longest visit, shortest visit), insurance billing history, procedural history, procedural and/or practice specialties, provider quality metric (e.g., based on quality of service (e.g., based on feedback from members, professional organizations, awards earned), claim submissions (e.g., submitted on time with limited errors), percentage of patients associated with the service provider, use and management of the service provider application (e.g., clinical assessments, submitting referrals), ease of scheduling, delays associated with scheduling, etc.), and other information associated with the medical provider.

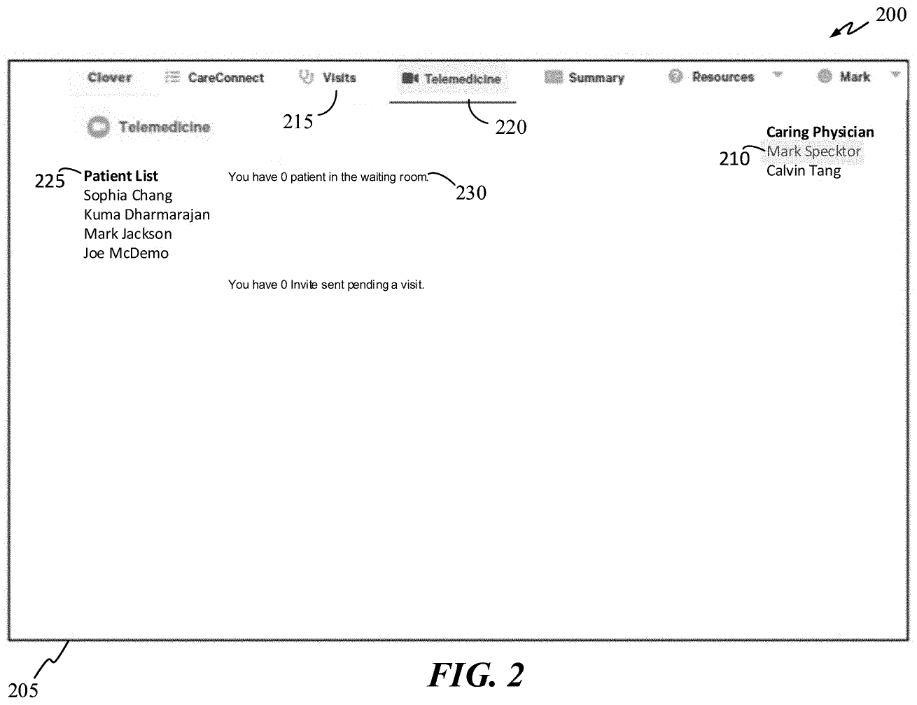

On a high level, healthcare provider (HP) 130 (e.g., nurses, doctors) can initiate a telehealth session with patient 152 by using one of the devices (e.g., 112, 114, 120) on HP side 110. For example, HP 130 can use laptop 120 to establish a telehealth session with patient 152 using a HP-side application (see FIG. 2) residing on laptop 120. The HP-side application can be an internet browser (e.g., Chrome, MS Edge), extension for an internet browser, a standalone application, or a mobile app where the computing device is a mobile device such as an iPhone, a tablet, or an Android-based device.

Prior to establishing the telehealth session, HP 130 can use the HP-side application to send an invitation to patient 152 requesting patient 152 to attend the telehealth session. The invitation can be sent via SMS to patient 152 mobile phone, email, or other messaging applications (e.g., Messenger, WeChat). Alternatively, patient 152 has already received a telehealth session invitation during an earlier appointment stage via email or SMS. Patient 152 can accept the invitation to the telehealth session, which can immediately establish the telehealth session between patient 152 and HP 130. Alternatively, upon acceptance of the invitation, patient 152 is placed in a virtual waiting room until HP 130 activate the telehealth session when HP 130 becomes available. In practice, HP 130 can have multiple patients in the virtual waiting room queued by their appointment time. However, the patients in the waiting room are still required to wait for HP 130 to activate the telehealth session.

The HP-side application can either directly communicate with patient device 155 over the internet using a communication protocol such as, but not limited to, voice over IP. The HP-side application can also communicate with patient device 155 over wireless communication network such as the 3G, 4G, or 5G cellular network. Alternatively, a server-side telehealth application residing on a remote server can manage and/or host the telehealth session between the HP-side application of an HP device and device 155 of patient 152. In this way, the HP-side application does not have to store and mange patient-related data such as contact information, device identifier (e.g., IMEI) of device 155, medical records, appointment history, upcoming appointments, etc. In some embodiments, the server-side telehealth application can facilitate the telehealth session by establishing the communication connection between a device on HP-side 110 (e.g., 112-120) and device 155. The server-side telehealth application can manage the patient queue list for HP 130 by checking an appointment database (not shown) residing on server 170 to populate the queue with patient's data for display on the HP-side device. The telehealth application can also send one or more portions of the patient's data to device 155 for display such as lab results, previous diagnosis, last appointment, future appointments, prescriptions, etc.

In accepting the invitation for the telehealth session and/or establishing the telehealth session, patient 152 can use a patient-side application such as an internet browser (e.g., Chrome, MS Edge), an application extension (e.g., Chrome extension), or a standalone application. For example, the invitation can be in the form of a link to a website (which opens an internet browser) or to open a previously installed application (e.g., Clover's telehealth application) on device 155. For instance, the application can be a Clover Health telemedicine application that can be downloaded from an online App store for various platforms such as, but not limited to, Apple's IOS platform and Google's Android platform.

Once the telehealth session is established between HP 130 and patient 152, some of the patient-related data (e.g., personal info, medical records, insurance record, appointments, prescriptions) can be shown on the HP-side device (e.g., laptop 120) and/or device 155. In some embodiments, only HP 130 can make changes to the patient-related data. This can be done using GUIs generated by the HP-side application, which are displayed on the display of the HP-side device. Any changes made to one or more portions of the patient-related data can be saved and stored locally and/or remotely. In some embodiments, patient-related data are stored on server 170, which can be accessed by any healthcare providers at any locations in the world, assuming they are part of telehealth system 100.

In some embodiments, telehealth system 100 can authenticate, connect, and/or qualify a third-party device (e.g., device 157) on the patient-side to obtain one or more health characteristics such as, but not limited to, heart rate, breathing pattern, body temperature, weight, body mass index, EEG, and EKG. In this way, HP 130 can leverage the functionalities and capabilities of device 157 to obtain additional health data that can help with the diagnosis of the patient. For example, in order to qualify third-party device 157, patient 152 may be required to bring device 157 to an in person appointment for a calibration and/or validation test. For instance, device 157 can be used to measure the heart rate of the patient. The results from device 157 during the in-person office visit can then be compared with a heart rate obtained manually or from a previously vetted device at the medical office. Once device 157 is qualified, the device identifier can then be stored as an approved third-party device for measuring health data in future telehealth sessions.

Alternatively, device 157 can be provided by the healthcare provider and is pre-qualified for use in telehealth settings. During a telehealth session, device 157 can be authenticated and communicatively coupled to device 155 via a wireless communication protocol such as Bluetooth or internet protocol over a local area network (e.g., WiFi). The patient-side application can include an I/O communication module that enables the patient-side application to send instructions to and receive data from device 157. The patient-side application can graphically display data received from device 157 on a display of device 155. The patient-side application can also forward the data from device 157 to server 170 and/or to the HP-side device such as laptop 120. Once the data from device 157 is received by the HP-side application, the data can be graphically displayed on a display of a HP-side device. Data from device 157 can also be received and stored by server-side telehealth application residing on server 170 as part of patient's 152 medical records, which can be retrieved and displayed at a later time.

In some embodiments, patient-side application of device 155 is configured to allow the HP-side application to control device 157 by forwarding instructions from the HP-side application to device 157. In this way, HP 130 can actively control device 157 to change one or more settings and/or to make one or more measurements.

FIG. 2 illustrates an example screen 205 of the HP-side application 200 in accordance with some embodiments of the present disclosure. View 205 of HP-side application 200 shows HP 210 already logged into telehealth system 100. Once logged in, HP-side application 200 can retrieve HP 210 information such as patient list, appointment list, etc. HP 210 can view upcoming in-person appointments by selecting icon 215 and telehealth appointments by selecting icon 220. Although not shown, HP 210 can also view all upcoming in-person and telehealth appointments simultaneously.

In some embodiments, upcoming appointments for telehealth sessions can be displayed in window 225, which can be a list patients of today's telehealth appointments. Window 225 can also display the date and time of each appointment, primary reason for the appointment, and the expected/booked duration of the appointment, etc. HP-side application 200 can also show the number of patients in the waiting room waiting to be admitted to the live telehealth session with HP 130. Patients in the waiting room are patients that have already accepted the telehealth session invitation via email or text or has already entered the waiting room by entering it via a scheduling function of the patient-side telehealth application. For example, patient 152 can open the patient-side telehealth application on device 155 on the day of the telehealth session appointment. Once the patient-side telehealth application on device 155 is opened, the telehealth session appointment and a graphical trigger that triggers the telehealth session to be activated will be displayed.

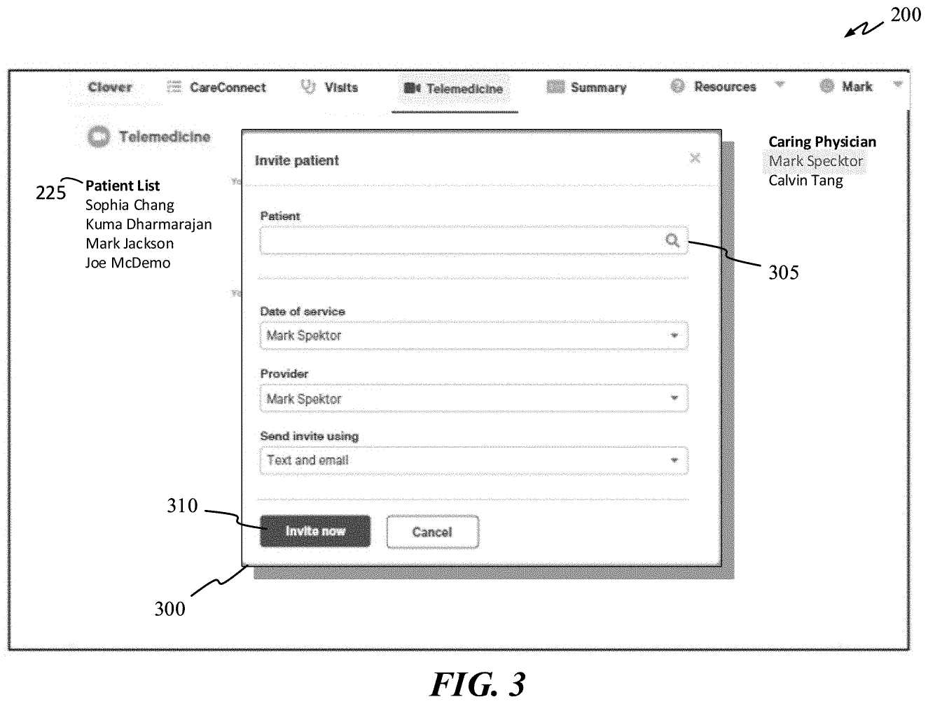

FIG. 3 illustrates an example screen of a patient invite window 300 of HP-side application 200 in accordance with some embodiments of the present disclosure. Using window 300 of HP-side application 200, HP 130 can find a patient and send the patient an invite to join a telehealth session at a scheduled time. A patient search field 305 enable HP 130 to search for any patient within the telehealth system 100 platform. Alternatively, search field 305 is configured to only search for patients assigned to one or more HPs or local branch. In this way, the search can be expedited. Once the patient's name is found, HP 130 can send an invite to the patient by selecting the invite now button 310.

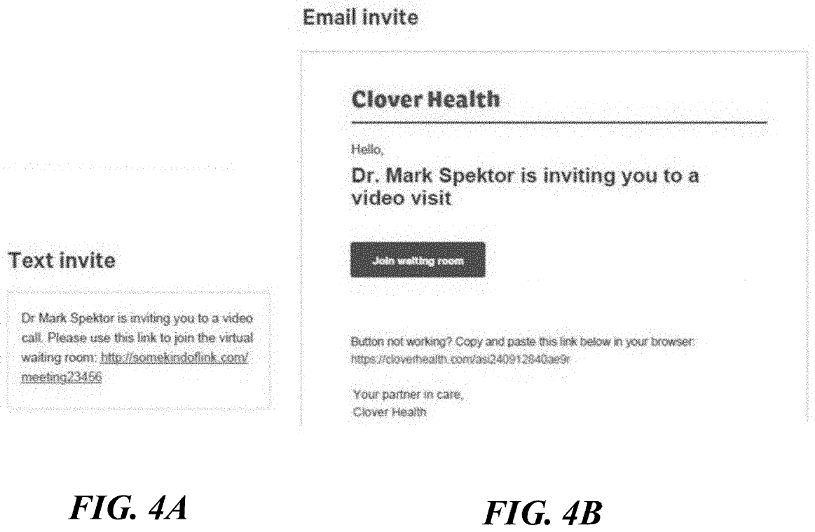

FIGS. 4A and 4B illustrate example invitations being sent to patient 152 after HP 130 select the invite button 310. FIG. 4A illustrates an example SMS invitation with a link to join the virtual waiting room. FIG. 4B illustrates an example email invitation with a link to join the virtual waiting room. The invitation can be sent to patient 152 well ahead of time (i.e., days ahead), but the link can lead to an inactive link or an inactive room until near the time of the scheduled telehealth session. When patient 152 selects the link near the scheduled telehealth session, patient 152 will be placed into a virtual waiting room until HP 130 actually activate the live the telehealth session.

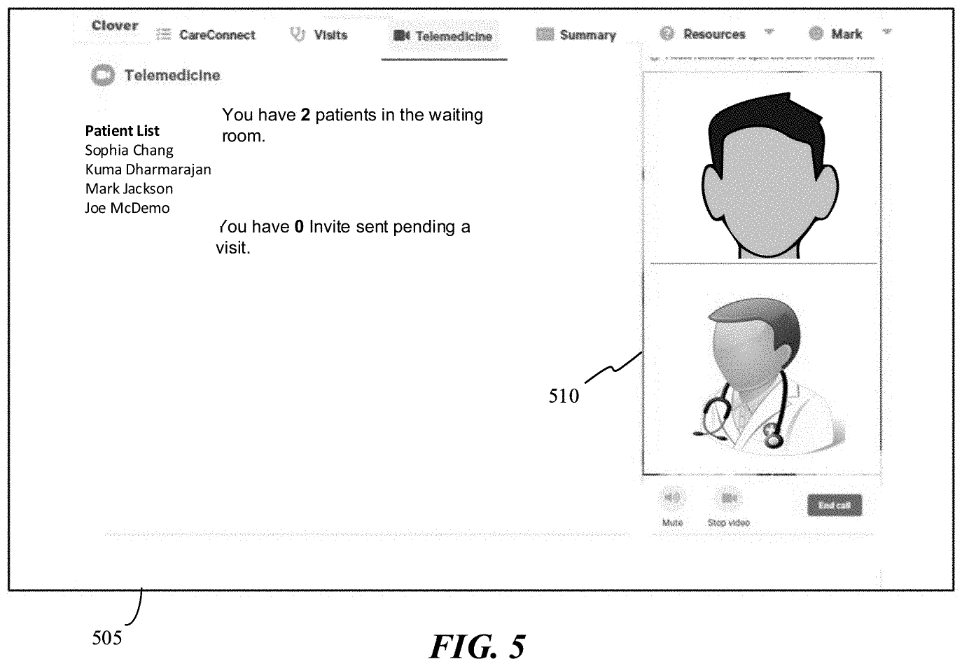

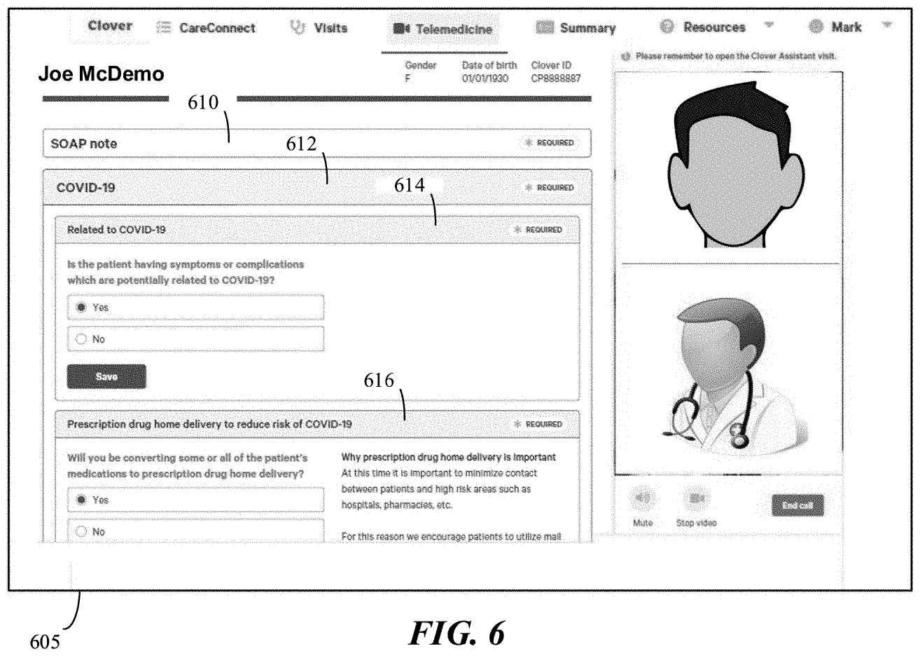

FIG. 5 illustrates an example screen 505 of HP-side application 200 in accordance with some embodiments of the present disclosure. Screen 505 includes a live telehealth session view 510 showing the live camera feed of HP 130 and patient 152. From screen 505, HP 130 can pull up patient-related data and display them on the left side of screen 505, which is shown in FIG. 6.

FIG. 6 illustrates an example screen 605 of HP-side application 200 in accordance with some embodiments of the present disclosure. Screen 605 includes a plurality of GUIs 610, 612, 614, 616 that can expand and display various patient-related data such as, but not limited to, medical records, current diagnosis, prescription information, and patient's special requirements. HP 130 can select each of GUIs 610, 612, 614, and 616 to expand and display more information. The data displayed in each of the GUIs can be modified by HP 130.

In some embodiments, HP 130 can also select a GUI and share the information with patient 152. For example, HP 130 can select GUI 610 and have the information in GUI 610 be transmitted to and display on device 155 by selecting a share with patient function (not shown). Although screen 605 only shows GUIs 610 through 616, there can be many more GUIs, which are revealed one at a time when HP 130 scroll down the left portion of screen 605. In some embodiments, HP 130 can highlight one or more portions a GUI and share only the selected highlighted one or more portions with patient 152 when the share function is selected.

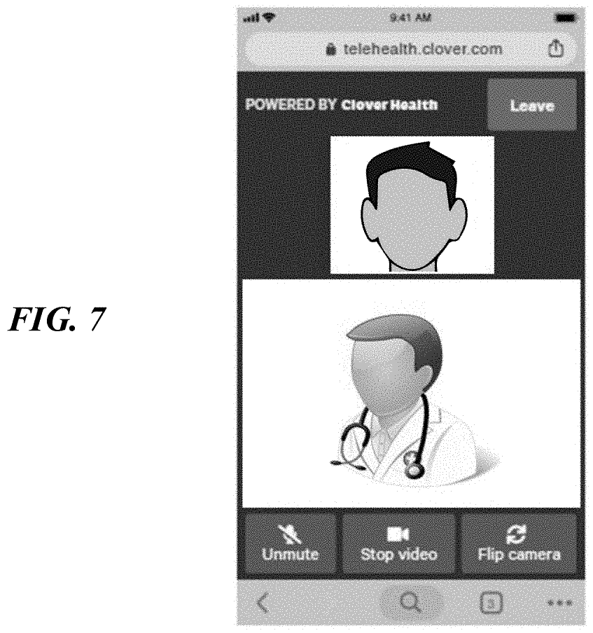

FIG. 7 illustrates an example telehealth session screen 705 shown on display 155. Screen 705 can be rendered by the patient-side application. Screen 705 can display the HP's name or other identifying information. Screen 705 can also display one or more patient-related data such as current heart rate, lab results, or any other data HP 130 wants to share with patient 152.

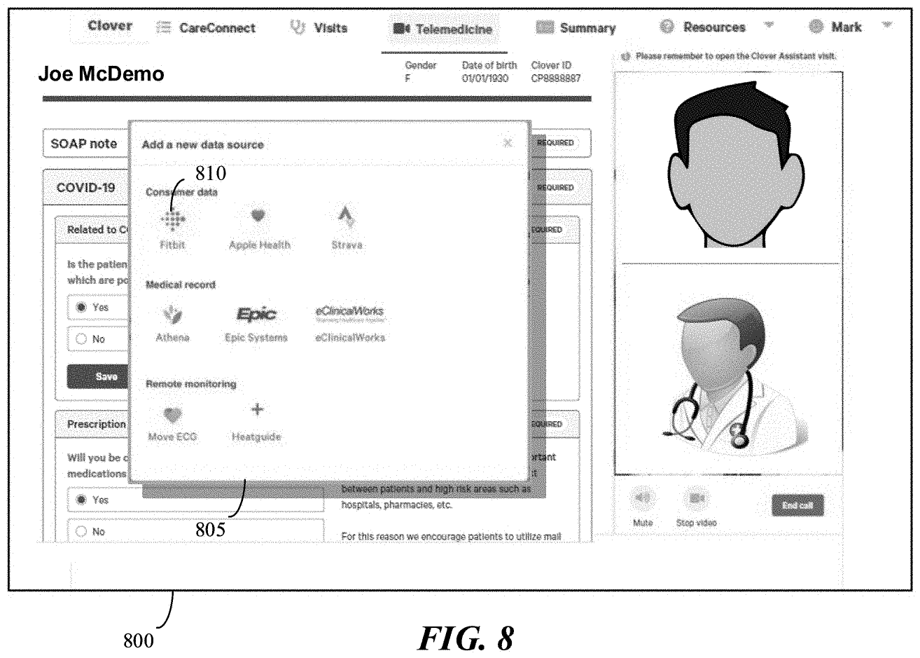

FIG. 8 illustrates a screen 800 HP-side application 200 in accordance with some embodiments of the present disclosure. Screen 800 includes a new data source window 805 that enables HP 130 to select additional data source from one or more external data sources such as servers 170 or live streaming data collected from third-party devices such as device 157. As shown, using window 805, HP 130 can add new data source from Fitbit, Apple Health, or other health monitoring devices such as a heart rate monitor or a glucose reader.

For example, when HP 130 selects the Fitbit icon 810 (or a health watch icon), HP-side application can send instructions to the patient-side application to connect with and authenticate the health watch device (e.g., Fitbit's watch, Apple watch, Android watch) that is in patient's 152 possession. The health watch (e.g., device 157) device can be previously authenticated and qualified. Alternatively, the health watch device can be a previously authenticated and qualified device given to patient 152 by the healthcare provider. Once the patient-side application successfully connects with the health watch, it can relay instructions and data between the health watch and the HP-side application and/or the telehealth application residing on server 170. In some embodiments, all data collected by health watch are collected and store on server 170 for later diagnosis. The data collected by health watch can also be transmitted a HP-side device (e.g., laptop 120) so that the data can be graphically displayed on the HP-side device. In this way, HP 130 can make better diagnosis of patient 152 health status.

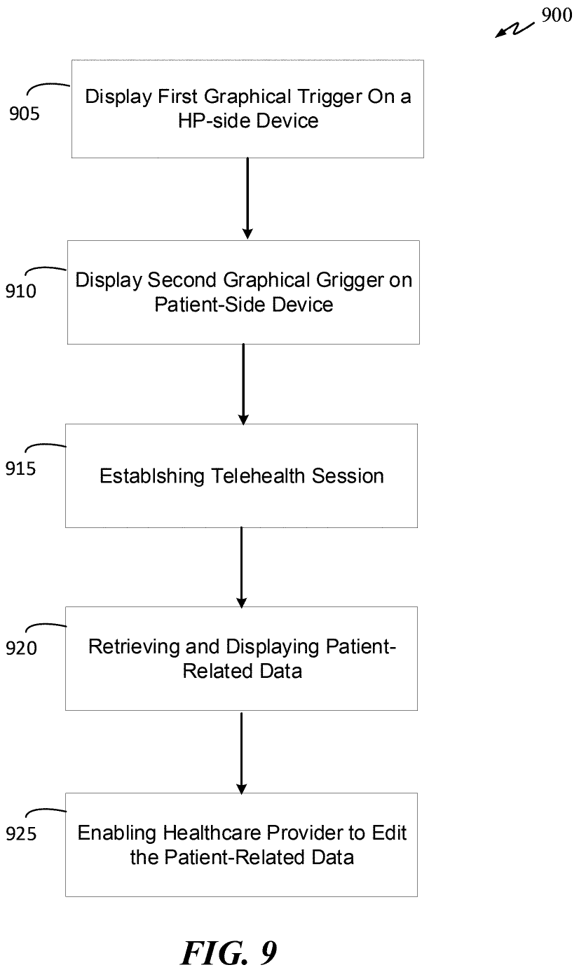

FIG. 9 illustrates a process 900 for establishing a telehealth session in accordance with some embodiments of the present disclosure. Process 900 starts at 905 where a graphical trigger is displayed on a HP-side device such as laptop 120. The graphical trigger can be the “invite now” button 310, which is displayed on laptop 120 once HP 130 selects a patient from a list of patients to schedule a telehealth session. As previously mentioned, once the invitation is sent, patient 152 can receive an invite via SMS and/or email. Alternatively, an alert can be sent to a patient-side telehealth application residing on the patient's device (e.g., device 155) alerting patient 152 that his/her physician has scheduled a telehealth session with the patient.

In some embodiments, in response to the HP 130 sending the invitation to a telehealth session, the patient-side device is configured to display a graphical trigger on a display of the patient-side device (e.g., device 155) to alert patient 152 that he/she has received an invite for a telehealth session with HP 130. The invitation can be for an instant telehealth session or for a future timeframe. The graphical trigger on the patient-side device can be a link to the virtual waiting room. The link can be part of the SMS, email, or a notification (e.g., push update) through the telehealth application installed on the patient-side device. In some embodiments, the patient-side graphical trigger, once selected, is configured to establish a live telehealth session with HP 130 instead of being directed to a waiting room (915).

At 915, to establish a live telehealth session with one of the patients in the waiting room, HP 130 can select the next patient in the queue and enable the live telehealth session by selecting the admit button, for example. Once the patient is admitted, the HP-side telehealth application or the server-side telehealth application can retrieve the patient's patient-related data, which can be displayed on the HP-side device. In some embodiments, HP 130 can use the GUI of the HP-side telehealth application to select one or more portions of the patient-related data to share with the patient. For example, HP 130 can select the previous lab results displayed on the HP-side device and share it with the patient by selecting, for example, a share function. This would cause the HP-side telehealth application and/or the server-side telehealth application to send the selected portion of the patient-related data to the patient-side device (e.g., device 155) for display. In this way, HP 130 has full control of what patient 152 can see and there can better drive and control the telehealth session.

At 925, using one or more GUIs of the HP-side telehealth application, HP 130 can make edit, delete, add to one or more portions of the patient-related data. Any changes made will be saved and stored by server 170.

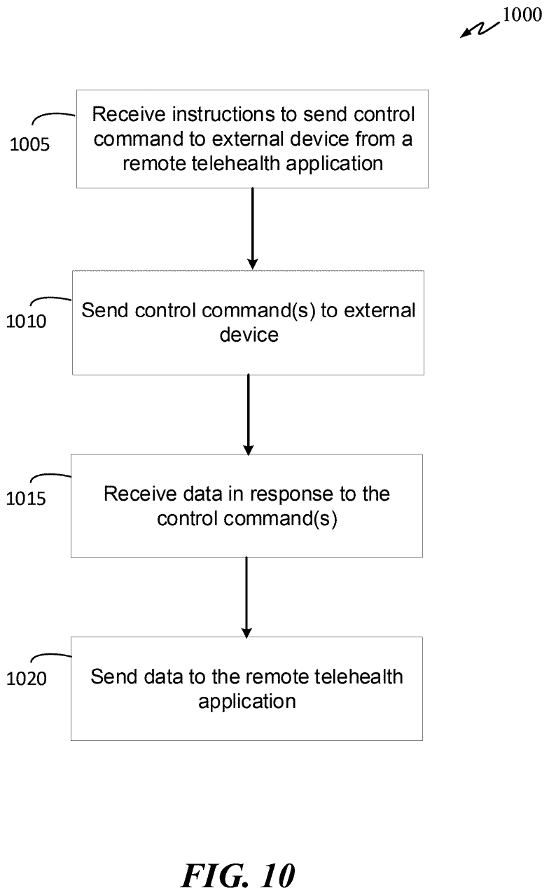

FIG. 10 illustrates a process 1000 for controlling an external device via the patient-side telehealth application in accordance with some embodiments of the present disclosure. Process 1000 enables HP 130 to remotely control an external device communicatively connected to the patient-side device (e.g., device 155). The external device can be health watch configured to measure various physical characteristics such as heart rate, blood, pressure, temperature, EEG, etc. The external device can also be a heart rate monitor, a thermometer, a scale, a BMI scale, an EKG, etc. First, the external device is communicatively paired with the patient-side device using a communication protocol such as, but not limited to, the Bluetooth communication protocol. This enables the patient-side to communicate with and control the external device (e.g., health watch 157).

At 1005, instructions to send control one or more commands (e.g., perform measurement, reset, change a measurement setting or specification) to the external device is received by the patient-side device via the patient-side telehealth application. The instructions to send a control command can be received from a remote telehealth application residing on the HP-side device (e.g., laptop 120) or server 170. At 1010, the patient-side application on the patient-side device is configured to forward the one or more control commands to the external device using Bluetooth or other suitable communication protocols (e.g., NFC).

At 1015, in response to sending the control command(s) to the external device, data is received from the external device at the patient-side device. At 1020, the patient-side telehealth application is configured to forward the data collected from the external device directly to the HP-side device and/or to the server-side telehealth application residing on server 170. In this way, HP 130 can view in almost real-time the health characteristics of patient 152 being measured and monitored by the external device. In some embodiments, upon receiving the health characteristics data from the external device, the HP-side telehealth application can display the data on the display of the HP-side device.

### Additional Example Embodiments

In a first example method for controlling a health monitoring device from a remote telehealth application during a live telehealth session, the method includes: receiving, at a local device, one or more commands configured to control the health monitoring device from the remote telehealth application, wherein the local device is communicatively paired with the health monitoring device; sending, at the local device, the one or more commands to the health monitoring device; in response to sending the one or more commands, receiving health data of a patient at the local device; sending, by the local device, the health data to the remote telehealth application; and displaying the health data on a display of a remote device.

In the first example method, the remote telehealth application resides on a remote server. The remote telehealth application can also reside on the healthcare provider device such as a computer or a mobile device. In an aspect of the first example method, the local device is paired with the health monitoring device using the Bluetooth communication standard or other suitable communication standards such as NFC.

In another aspect of the first example method, the one or more commands comprise one of a commence measuring health data command, a reset command, and a command to change one or more of the health monitoring device settings.

### Example System Architecture

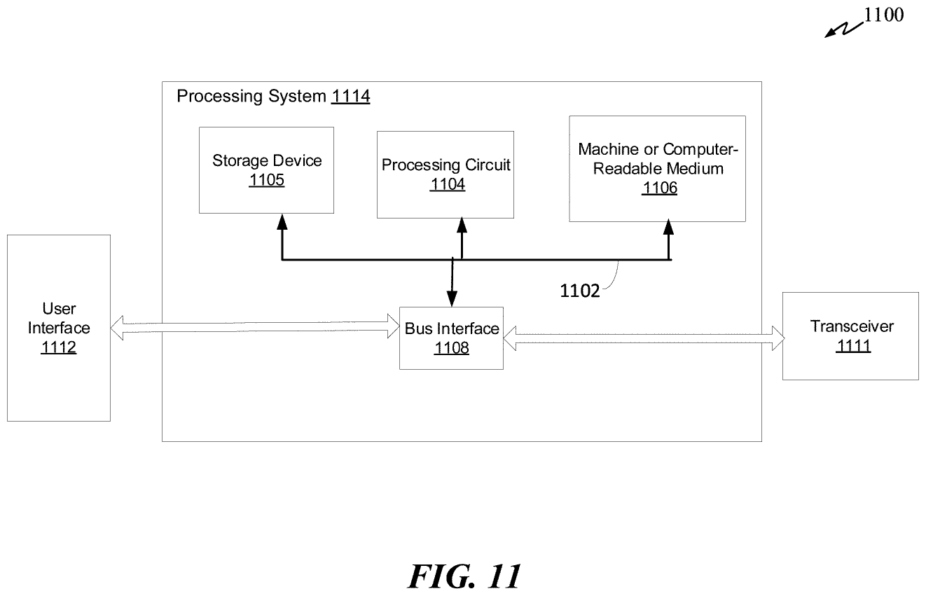

FIG. 11 illustrates an example system or apparatus 1100 in which telehealth application 200 (FIGS. 2 through 8) and processes 900 and 1000 can be implemented. In accordance with various aspects of the disclosure, an element, or any portion of an element, or any combination of elements may be implemented with a processing system 1114 that includes one or more processing circuits 1104. Processing circuits 1104 may include micro-processing circuits, microcontrollers, digital signal processing circuits (DSPs), field programmable gate arrays (FPGAs), programmable logic devices (PLDs), state machines, gated logic, discrete hardware circuits, and other suitable hardware configured to perform the various functionalities described throughout this disclosure. That is, the processing circuit 1104 may be used to implement any one or more of the processes described above and illustrated in FIGS. 9 and 10.

In FIG. 11, the processing system 1114 may be implemented with a bus architecture, represented generally by the bus 1102. The bus 1102 may include any number of interconnecting buses and bridges depending on the specific application of the processing system 1114 and the overall design constraints. The bus 1102 may link various circuits including one or more processing circuits (represented generally by the processing circuit 1104), the storage device 1105, and a machine-readable, processor-readable, processing circuit-readable or computer-readable media (represented generally by a non-transitory machine-readable medium 1106). The bus 1102 may also link various other circuits such as, but not limited to, timing sources, peripherals, voltage regulators, and power management circuits, which are well known in the art, and therefore, will not be described any further. The bus interface 1108 may provide an interface between bus 1102 and a transceiver 1110. The transceiver 1110 may provide a means for communicating with various other apparatus over a transmission medium. Depending upon the nature of the apparatus, a user interface 1112 (e.g., keypad, display, speaker, microphone, touchscreen, motion sensor) may also be provided.

The processing circuit 1104 may be responsible for managing the bus 1102 and for general processing, including the execution of software stored on the machine-readable medium 1106. The software, when executed by processing circuit 1104, causes processing system 1114 to perform the various functions described herein for any particular apparatus. Machine-readable medium 1106 may also be used for storing data that is manipulated by processing circuit 1104 when executing software.

One or more processing circuits 1104 in the processing system may execute software or software components. Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise. A processing circuit may perform the tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory or storage contents. Information, arguments, parameters, data, etc. may be passed, forwarded, or transmitted via any suitable means including memory sharing, message passing, token passing, network transmission, etc.

The software may reside on machine-readable medium 1106. The machine-readable medium 1106 may be a non-transitory machine-readable medium. A non-transitory processing circuit-readable, machine-readable or computer-readable medium includes, by way of example, a magnetic storage device, an optical disk, a memory module, a smart card, a flash memory device, and any other suitable medium for storing software and/or instructions that may be accessed and read by a machine or computer. The terms “machine-readable medium”, “computer-readable medium”, “processing circuit-readable medium” and/or “processor-readable medium” may include, but are not limited to, non-transitory media such as, but not limited to, portable or fixed storage devices, optical storage devices, and various other media capable of storing, containing or carrying instruction(s) and/or data. Thus, the various methods described herein may be fully or partially implemented by instructions and/or data that may be stored in a “machine-readable medium,” “computer-readable medium,” “processing circuit-readable medium” and/or “processor-readable medium” and executed by one or more processing circuits, machines and/or devices. The machine-readable medium may also include, by way of example, a carrier wave, a transmission line, and any other suitable medium for transmitting software and/or instructions that may be accessed and read by a computer.

The machine-readable medium 1106 may reside in the processing system 1114, external to the processing system 1114, or distributed across multiple entities including the processing system 1114. The machine-readable medium 1106 may be embodied in a computer program product. By way of example, a computer program product may include a machine-readable medium in packaging materials. Those skilled in the art will recognize how best to implement the described functionality presented throughout this disclosure depending on the particular application and the overall design constraints imposed on the overall system.

Throughout this disclosure, the preferred embodiment and examples illustrated should be considered as exemplars, rather than as limitations on the present inventive subject matter, which includes many inventions. As used herein, the term “inventive subject matter,” “system,” “device,” “apparatus,” “method,” “present system,” “present device,” “present apparatus” or “present method” refers to any and all of the embodiments described herein, and any equivalents.

It should also be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

When an element or feature is referred to as being “on” or “adjacent” to another element or feature, it can be directly on or adjacent the other element or feature or intervening elements or features may also be present. In contrast, when an element is referred to as being “directly on” or extending “directly onto” another element, there are no intervening elements present. Additionally, when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Furthermore, relative terms such as “inner,” “outer,” “upper,” “top,” “above,” “lower,” “bottom,” “beneath,” “below,” and similar terms, may be used herein to describe a relationship of one element to another. Terms such as “higher,” “lower,” “wider,” “narrower,” and similar terms, may be used herein to describe angular relationships. It is understood that these terms are intended to encompass different orientations of the elements or system in addition to the orientation depicted in the figures.

Although the terms first, second, third, etc., may be used herein to describe various elements, components, regions, and/or sections, these elements, components, regions, and/or sections should not be limited by these terms. These terms are only used to distinguish one element, component, region, or section from another. Thus, unless expressly stated otherwise, a first element, component, region, or section discussed below could be termed a second element, component, region, or section without departing from the teachings of the inventive subject matter. As used herein, the term “and/or” includes any and all combinations of one or more of the associated list items.

The terminology used herein is for the purpose of describing particular embodiments only and is not intended to be limiting. As used herein, the singular forms “a,” “an,” and “the” are intended to include the plural forms as well, unless the context clearly indicates otherwise. For example, when the present specification refers to “an” assembly, it is understood that this language encompasses a single assembly or a plurality or array of assemblies. It will be further understood that the terms “comprises,” “comprising,” “includes,” and/or “including” when used herein, specify the presence of stated features, integers, steps, operations, elements, and/or components, but do not preclude the presence or addition of one or more other features, integers, steps, operations, elements, components, and/or groups thereof.

Embodiments are described herein with reference to view illustrations that are schematic illustrations. As such, the actual thickness of elements can be different, and variations from the shapes of the illustrations as a result, for example, of manufacturing techniques and/or tolerances are expected. Thus, the elements illustrated in the figures are schematic in nature and their shapes are not intended to illustrate the precise shape of a region and are not intended to limit the scope of the inventive subject matter.

The foregoing is intended to cover all modifications, equivalents and alternative constructions falling within the spirit and scope of the invention as expressed in the appended claims, wherein no portion of the disclosure is intended, expressly or implicitly, to be dedicated to the public domain if not set forth in the claims. Furthermore, any features, functions, steps, or elements of the embodiments may be recited in or added to the claims, as well as negative limitations that define the inventive scope of the claims by features, functions, steps, or elements that are not within that scope.

What is claimed is:

1. A system for establishing a telehealth session between a patient and a healthcare provider (HP), the HP using a first device, the patient using a second device, the system comprising:
a HP-side application executable on one or more processors of the first device, the HP-side application configured to display a first graphical trigger on a display of the second device that when selected causes the first device to send a connection request to communicatively connect with the patient over the telehealth session, wherein the HP-side application does not have access to contact information of the second device;
a telehealth application executable on one or more processors of a server, the telehealth application configured to receive, from the first device, the connection request to communicatively connect with the patient, in response to receiving the connection request from the first device, the telehealth application is configured to:
determine a contact information of the second device; and
send instructions to the second device based on the contact information of the second device; and
a patient-side application executable on one or more processors of the second device, the patient-side application configured to display a second graphical trigger on a display of the second device in response to receiving the instructions from the telehealth application, wherein the second graphical trigger, when selected, causes the patient-side application of the second device to communicatively connect with the HP-side application of the first device to establish the telehealth session;
wherein the HP-side application is further configured to:
generate one or more graphical user interfaces (GUIs) that display patient-related data on the display of the first device during the telehealth session;
enable the healthcare provider to edit, using the one or more GUIs, one or more portions of the patient-related data during the telehealth session; and
send the one or more edited portions to the telehealth application.

2. The system of claim 1, wherein the HP-side application is further configured to:
display a list of patients on the display of the first device, wherein each patient on the list of patients is selectable;
receive a selection of the patient from the list of patients;
send information relating to the selected patient to the telehealth application; and
receive patient-related data of the selected patient from the telehealth application.

3. The system of claim 2, wherein the HP-side application is further configured to:
obtain identifying information of the HP; and
receive a list of patients based on the identifying information of the HP.

4. The system of claim 1, wherein the telehealth application is configured to establish the telehealth session between the first and second devices in response to receiving information indicating that the second graphical trigger is selected.

5. The system of claim 1, wherein the patient-side application is configured to connect to a virtual waiting room hosted by the telehealth application on the server prior to the telehealth session with the HP-side application of the first device is established by the telehealth application.

6. The system of claim 5, wherein the telehealth application is configured to queue the patient-side application until the patient's scheduled appointment time and to establish the telehealth session between the first and second devices once scheduled appointment time is reached, and to request the healthcare provider, using the HP-side client, to confirm moving the patient from the virtual waiting room to the telehealth session.

7. The system of claim 1, wherein the patient-side application is configured to:
authenticate a health monitoring device;
receive one or more types of health data from the health monitoring device during the telehealth session; and
sending the one or more types of health data to the second device.

8. The system of claim 7, wherein the patient-side application is configured to display the one or more types of health data on the display of the second device.

9. The system of claim 7, wherein the HP-side application is configured to receive and display the one or more types of health data on the display of the first device.

10. The system of claim 7, wherein the patient-side application is configured to enable the HP-side application to remotely control the health monitoring device.

11. The system of claim 1, wherein the second device and the server are same device.

12. The system of claim 1, wherein patient-related data comprise personal and health data of a patient.

13. A system for establishing a telehealth session between a patient and a healthcare provider (HP), the HP using a first device, the patient using a second device, the system comprising:
a HP-side application executable on one or more processors of the first device, the HP-side application configured to display a first graphical trigger on a display of the first device that when selected causes the first device to send a connection request to establish a telehealth session with the patient on the second device,
a patient-side application executable on one or more processors of the second device, the patient-side application configured to display a second graphical trigger on a display of the second device in response to receiving the connection request from the first device, wherein the second graphical trigger, when selected, causes the patient-side application of the second device to communicatively connect with the HP-side application of the first device to establish the telehealth session;
wherein the HP-side application is further configured to:
generate one or more graphical user interfaces (GUIs) that display patient-related data on the display of the second device during the telehealth session;
enable the healthcare provider to edit, using the one or more GUIs, one or more portions of the patient-related data during the telehealth session; and
send the one or more edited portions of the patient-related data to the telehealth application.

14. A computer-implemented method for establishing a telehealth session between a patient and a healthcare provider (HP), the HP using a first device, the patient using a second device, the method comprising:
displaying a first graphical trigger on a display of the first device that when selected causes the first device to send a connection request to communicatively connect with the patient over the telehealth session with the second device;
in response to the connection request, displaying a second graphical trigger on a display of the second device, wherein the second graphical trigger, when selected, causes the telehealth session to be established between the first and second devices;
generating one or more graphical user interfaces (GUIs) that display patient-related data on the display of the first device during the telehealth session;
enabling the HP to edit, using the one or more GUIs, one or more portions of the patient-related data during the telehealth session; and
saving the one or more edited portions of the patient-related data.

15. The computer-implemented method of claim 14, further comprising:
receiving, at a remote server, the connection request to communicatively connect with the patient over the telehealth session with the second device;
retrieving, at the remote server, identifying information of the second device and patient-the related data;
sending, by the remote server, instructions to establish the telehealth session to the second device using the identifying information;
receiving information indicating that the second graphical trigger is selected; and
establishing, by the remote server, the telehealth session between the first and second devices in response to receiving information indicating that the second graphical trigger is selected.

16. The computer-implemented method of claim 15, wherein establishing the telehealth session between the first and second devices. Comprises in response to receiving information indicating that the second graphical trigger is selected.

17. The computer-implemented method of claim 14, further comprising:
displaying a list of patients on the display of the first device, wherein each patient on the list of patients is selectable;
receiving a selection of a first patient from the list of patients; and
sending information relating to the selected patient to the telehealth application.

18. The computer-implemented method of claim 14, further comprising:
receiving, at the remote server, identifying information of the HP from the first device;
determining, at the remote server, a list of patients scheduled to meet with the HP based on the identifying information of the HP; and
sending the list of patient scheduled to meet with the HP to the first device for display.

19. The computer-implemented method of claim 14, further comprising connecting the second device to a virtual waiting room hosted by a remote server prior to the telehealth session between the first and second devices is established.

20. The computer-implemented method of claim 19, further comprising queuing the second device in the virtual waiting room until receiving a connection confirmation from the first device.

21. The computer-implemented method of claim 14, further comprising:
receiving, at the second device, one or more types of health data from a health monitoring device during the telehealth session; and
sending the one or more types of health data to the first device for display.

22. The computer-implemented method of claim 21, enabling the first device to remotely control the health monitoring device.
